(19) United States
(12) Patent Application Publication
(10) Pub. No.: US 2023/0371987 A1
(43) Pub. Date: Nov. 23, 2023

(54) IMPLANT WITH MULTI-MODAL ADJUSTMENT

(71) Applicant: NuVasive Specialized Orthopedics, Inc., San Diego, CA (US)

(72) Inventors: Dalton Jennings, Aliso Viejo, CA (US); Adam Beckett, Laguna Hills, CA (US); Fernando Cardenas, San Diego, CA (US); Johnny Chen, Aliso Viejo, CA (US); Jorge Lopez Camacho, Oxnard, CA (US); Nathan Meyer, Vista, CA (US); Wilfred Medel, San Diego, CA (US); Woong Kim, Fresno, CA (US); Gabriel Buenviaje, Laguna Hills, CA (US); Shawn Placie, Aliso Viejo, CA (US)

(21) Appl. No.: 18/140,915

(22) Filed: Apr. 28, 2023

Related U.S. Application Data
(60) Provisional application No. 63/342,921, filed on May 17, 2022.

(51) Int. Cl. A61B 17/72

(52) U.S. Cl. CPC A61B 17/72; A61F 2220/0025

(57) ABSTRACT

Various implementations include implants and related methods for moving bone. Certain implementations include an implant for moving bone in a patient's body, the implant including: an implantable biocompatible housing having a first cavity; a first adjustment rod at least partially contained within the first cavity; and a drive mechanism configured to drive the first adjustment rod to enable both translation axially relative to the housing and rotation about a primary axis of the housing.

### CROSS REFERENCE TO RELATED APPLICATION

The present patent application claims priority to U.S. Provisional Patent Application No. 63/342,921, filed on May 17, 2022. The foregoing application is incorporated by reference as though fully set forth herein.

### TECHNICAL FIELD

This disclosure generally relates to biocompatible implants. More particularly, the disclosure relates to implants for moving bone in a patient's body.

### BACKGROUND

Implantable bone adjustment systems can beneficially treat a variety of conditions. For example, implantable bone adjustment systems can be used for purposes of distraction osteogenesis (also known as distraction callotasis and osteodistraction) in applications such as: post osteosarcoma bone cancer; cosmetic lengthening (both legs-femur and/or tibia) in short stature or dwarfism/achondroplasia; lengthening of one limb to match the other (congenital, post-trauma, post-skeletal disorder, prosthetic knee joint), nonunions, etc. Additionally, implantable bone adjustment systems can be used in treatment of various additional conditions and ailments such as scoliosis or osteoarthritis (e.g., knee osteoarthritis). Additional examples of treatment applications for implantable bone adjustment systems are described in U.S. patent application Ser. No. 16/298,339 (filed Mar. 11, 2019) and U.S. patent application Ser. No. 13/370,966 (filed Feb. 14, 2011), which are incorporated herein by reference in their entirety.

### SUMMARY

The needs above, as well as others, are addressed by embodiments of implants and related methods described in this disclosure. All examples and features mentioned below can be combined in any technically possible way.

Various implementations include implants for moving bone in a patient's body, related systems, and methods. Certain implementations include a biocompatible implant configured for multi-modal adjustment of patient bone.

In particular aspects, an implant for moving bone in a patient's body includes: an implantable biocompatible housing having a first cavity; a first adjustment rod at least partially contained within the first cavity; and a drive mechanism configured to drive the first adjustment rod to enable both translation axially relative to the housing and rotation about a primary axis of the housing.

In additional particular aspects, an implant for moving bone in a patient's body includes: an implantable biocompatible housing having a first cavity and a second cavity; a set of adjustment rods at least partially contained within the first cavity and the second cavity; and a drive mechanism configured to drive the set of adjustment rods to enable both translation axially relative to the housing and rotation about a primary axis of the housing.

In additional particular aspects, a method of intramedullary adjustment of a patient's bone is performed using an implant described according to aspects of the disclosure.

Implementations may include one of the following features, or any combination thereof.

In particular aspects, the drive mechanism includes two distinct drivers configured to be actuated by an external control device.

In certain cases, the distinct drivers are coupled to the housing at distinct locations, a first one of the drivers controlling translation of the first adjustment rod and a second one of the drivers controlling rotation of the first adjustment rod.

In some implementations, the external control device includes an actuator for communicating with the drive mechanism from a location external to the patient's body.

In particular cases, the first adjustment rod includes at least one helical groove, and the housing includes a rotation control lug for engaging the at least one helical groove, such that for at least a portion of an axial extent of translation of the first adjustment rod, the rotation control lug causes the first adjustment rod to rotate about the primary axis.

In some cases, the rotation control lug includes at least one tab configured to mate with the at least one helical groove. In some examples, the rotation control lug includes two tabs for mating with two helical grooves.

In particular aspects, the at least one helical groove has a dimension defined by a patient adjustment profile for the patient's body. In some examples, the dimension defined by the patient adjustment profile is customized for the patient, including in some cases, groove diameter, pitch, and/or grooves per inch/cm.

In certain implementations, when driven by the drive mechanism, the first adjustment rod is configured to translate and rotate simultaneously.

In some cases, the first adjustment rod is configured to separately translate and rotate in response to driving by the drive mechanism. In certain examples, the first adjustment rod translates a first distance, and after first distance is reached, is free to rotate.

In particular aspects, the first adjustment rod includes a radially extending tab, and the housing includes a sleeve having an axially extending slot for engaging the radially extending tab.

In certain cases, the axially extending slot engages the radially extending tab to limit rotation along only a portion of the axial extent of translation of the first adjustment rod.

In some aspects, a proximal end of the first adjustment rod includes at least one rotation limiting feature for limiting rotation while the radially extending tab is not engaged with the axially extending slot in the sleeve.

In particular implementations, the first adjustment rod includes a cam member having a set of teeth for engaging complementary teeth in the housing.

In certain aspects, the first adjustment rod is rotationally limited by interaction of the set of teeth in the cam member with the complementary teeth. In some examples, the first adjustment rod is rotationally limited by a self-locking, or anti-back-rotation feature.

In some implementations, the set of complementary teeth in the housing are axially offset to enable incremental translation adjustment of the first adjustment rod. In certain examples, the incremental translation adjustment includes stepwise translation and/or rotation.

In particular cases, the drive mechanism includes at least one spring and a gear pack for controlling rotation of the first adjustment rod.

In certain aspects, the first adjustment rod includes a set of retractable tabs that complement at least one mating feature in the housing. In some examples, the retractable tabs can be actuated via one or more mechanisms, e.g., a switch can rotate a body to retract/extend tab(s), a solenoid can actuate retraction/extension of tabs, tabs can have a taper that retracts/extends with movement of an intermediary sleeve or body.

In some implementations, when extended, the set of retractable tabs are configured to engage the at least one mating feature in the housing to limit rotation of the first adjustment rod, and when retracted, the set of retractable tabs enable rotation of the first adjustment rod relative to the housing.

In particular aspects, the implant further includes an electronic controller coupled with the first adjustment rod to control at least one of retraction or extension of the set of retractable tabs, where the drive mechanism is configured to drive the axial translation of the first adjustment rod.

In certain cases, a proximal end of the first adjustment rod engages a lead screw and is configured to move with the lead screw, and while the retractable tabs engage the at least one mating feature, the first adjustment rod is configured to translate during movement of the lead screw, and while the retractable tabs are retracted the first adjustment rod is configured to rotate during movement of the lead screw.

In particular aspects, the drive mechanism includes a pulley plate and a gear pack. In some examples, the gear pack is driven magnetically.

In certain cases, the pulley plate is coupled with a proximal end of the first adjustment rod to control translation of the first adjustment rod.

In some implementations, the gear pack engages complementary teeth or gears on the pulley plate to enable rotation of the pulley plate and the first adjustment rod.

In particular aspects, the gear pack includes at least one belt coupled with an actuator for driving the gear pack.

In certain implementations, the drive mechanism includes a magnetic actuator configured to be actuated by a magnetic field external to the patient's body.

In some cases, the implant is configured for intramedullary placement in a patient.

In certain aspects, the implant is configured to aid in treatment of a limb length discrepancy or a bone defect in the patient's body.

In some cases, a method of intramedullary adjustment of a patient's bone is performed using an implant according to various implementations.

In particular aspects, the set of adjustment rods includes a first adjustment rod at least partially contained within the first cavity and a second adjustment rod at least partially contained within the second cavity.

In certain implementations, the drive mechanism includes a clutch implant configured to control adjustment of both the first adjustment rod and the second adjustment rod.

In some cases, the clutch implant is positioned coaxially with the first adjustment rod and the second adjustment rod.

In certain aspects, the clutch implant is positioned between the first adjustment rod and the second adjustment rod.

In particular implementations, the clutch implant includes a first gear set for controlling translation of the first adjustment rod and a second gear set for controlling rotation of the second adjustment rod.

In some aspects, the clutch implant includes at least one of a magnetic clutch or an electronic clutch configured to be actuated by an external control device.

In certain cases, the drive mechanism includes a magnetic actuator configured to be actuated by a magnetic field external to the patient's body.

In some cases, the drive mechanism is the sole drive mechanism of the implant.

In particular aspects, the drive mechanism is configured to be magnetically driven by an external device.

In certain implementations, the drive mechanism is configured to be powered by an implanted power source.

Two or more features described in this disclosure, including those described in this summary section, may be combined to form implementations not specifically described herein.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

The details of one or more implementations are set forth in the accompanying drawings and the description below. Other features, objects and benefits will be apparent from the description and drawings, and from the claims.

### BRIEF DESCRIPTION OF THE DRAWINGS

FIG. 1 shows a semi-transparent perspective view of an intramedullary implant according to various implementations.

FIG. 2 shows a cross-section of the implant of FIG. 1 according to various implementations.

FIG. 3 is a perspective view of an intramedullary implant according to various implementations.

FIG. 4 shows a cross-section of the implant of FIG. 3 according to various implementations.

FIG. 5 shows a perspective view of a portion of an implant according to various additional implementations.

FIG. 6 shows a perspective view of an intramedullary implant according to various implementations.

FIG. 7 shows a cross-section of the implant of FIG. 6 according to various implementations.

FIG. 8 shows a close-up perspective view of a portion of the implant in FIGS. 6 and 7 according to various implementations.

FIG. 9 shows a perspective view of a portion of an implant and associated screw according to various implementations.

FIG. 10 shows a perspective view of an implant according to various additional implementations.

FIG. 11 shows a cross-section of an additional implant according to various implementations.

FIG. 12 shows a partially transparent perspective view of an implant according to various additional implementations.

FIG. 13 illustrates a cross-section of an additional implant according to certain implementations.

FIG. 14 shows a perspective view of the implant in FIG. 13 according to various implementations.

FIG. 15 is a simplified side view of an implant according to certain implementations.

FIG. 16 is a simplified cross-section of the implant of FIG. 15 with a control system illustrated, according to various implementations.

FIG. 17 is a perspective cutaway view of a portion of an implant according to various implementations.

FIG. 18 is a cross-sectional view of the implant of FIG. 17 according to various implementations.

FIG. 19 is a perspective view of an implant according to various additional implementations.

FIG. 20 is a cross-section of the implant of FIG. 19, according to various implementations.

FIG. 21 is a close-up view of a portion of the cross-section of FIG. 19, according to various implementations.

It is noted that the drawings of the various implementations are not necessarily to scale. The drawings are intended to depict only typical aspects of the disclosure, and therefore should not be considered as limiting the scope of the implementations. In the drawings, like numbering represents like elements between the drawings.

### DETAILED DESCRIPTION

Despite numerous applications, conventional implantable bone adjustment systems are limited in the extent and modality of adjustment. In certain cases, multiple adjustments are made or multiple distinct modalities are used to adjust bone, for example, in terms of distraction and rotation. These conventional approaches can be unnecessarily complex, costly, ineffective, or some combination thereof.

This disclosure provides, at least in part, implants for moving bone in a patient's body, and methods that beneficially incorporate such implants to move bone. These implants enable multi-modal adjustment, which can reduce time and complications associated with adjustment procedures. The various disclosed implementations can improve patient outcomes when compared with conventional implantable adjusters. The disclosed implementations can provide adaptability in adjusting bone positioning, enhancing one or both of intraoperative and postoperative engagement with the device. The implant can provide an implantable biocompatible housing with at least one adjustment rod, and a drive mechanism that is configured to drive the adjustment rod(s) to enable both translation and rotation relative to the housing. When compared with conventional approaches, the multi-modal (e.g., translational and rotational) adjustment implants described according to various implementations provide an efficient and simplified mechanism for bone adjustment. The implants described according to various implementations can also reduce health risks for patients when compared with conventional approaches, for example, allowing a single implant to perform functions that might otherwise be performed by multiple implantable devices (with associated implant procedures) or surgical interventions. Further, challenges exist in providing the mechanisms for performing multi-modal adjustment while maintaining a small enough device for useful implantation.

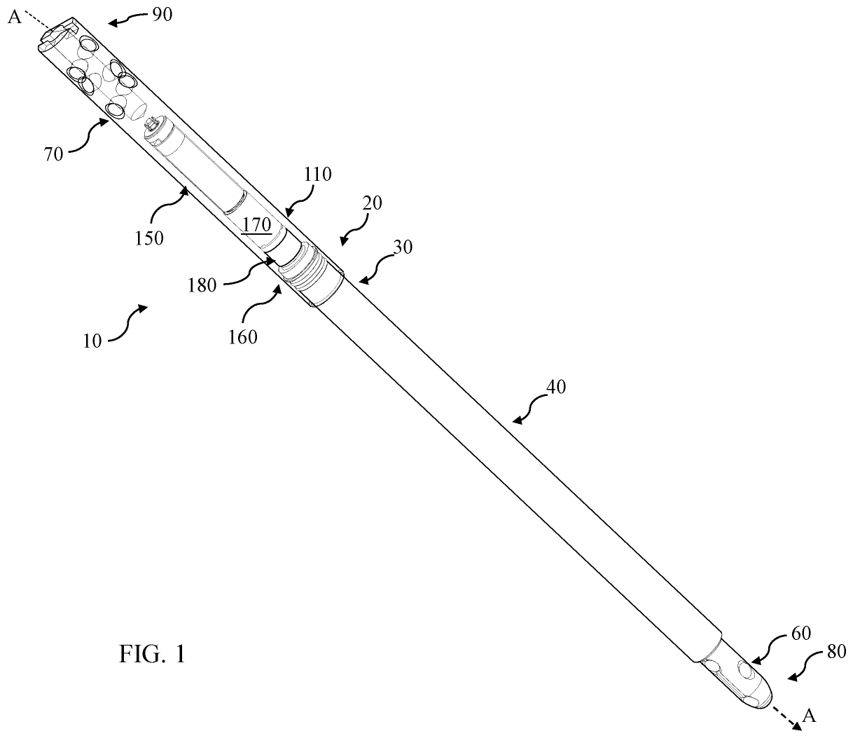

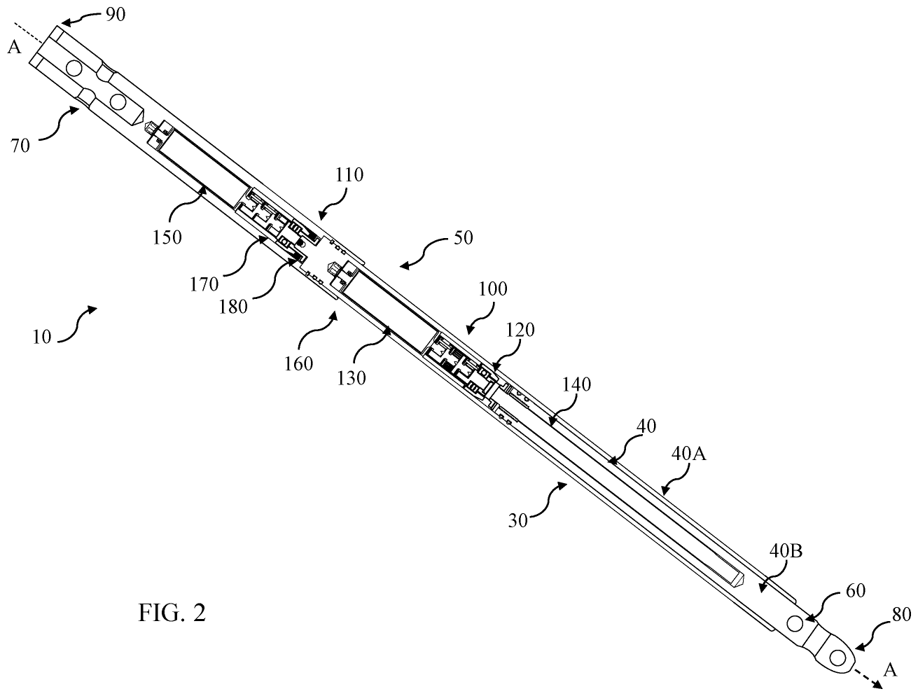

FIG. 1 shows a perspective view of an implant 10 for moving bone in a patient's body, and FIG. 2 shows a cross-sectional view of the implant 10. In particular implementations, the implant 10 (and other implants depicted and described herein) is configured for intramedullary placement in a patient, e.g., to aid in treatment of one or more patient conditions. In certain examples, the implant 10 can be used in a method of intramedullary adjustment of a patient's bone. In a particular example, the implant 10 (and other implant(s) herein) is configured to aid in treatment of a limb length discrepancy and/or a bone defect in the patient's body. In other implementations, the multi-modality techniques described herein can be applied in non-intramedullary uses, such as in the correction of scoliosis or for other uses.

In various implementations, the implant 10 includes an implantable biocompatible housing (or, “housing”) 20. The housing 20 can include a first cavity 30, with a first adjustment rod 40 at least partially contained in the first cavity 30. A drive mechanism 50 is configured to drive the first adjustment rod 40 to enable both translation (also called “distraction”) of the rod 40 axially relative to the housing 20 (relative to primary axis A), as well as rotation about the primary axis (A) of housing 20. The housing 20 and the first adjustment rod 40 are each configured for coupling to a patient. In various implementations, the first adjustment rod 40 includes one or more holes 60 for passing an anchor with which to secure the implant 10 to the patient (e.g., to bone). The housing 20 can also include one or more holes 70 for passing an anchor with which to secure the implant to the patient (e.g., to bone). Spacing of holes 60 relative to the distal end 80 of the implant 10, spacing of holes 70 relative to the proximal end 90 of the implant 10, dimensions of the holes 60, 70, and other dimensional aspects of the holes 60, 70 can be tailored for particular patient parameters and/or treatment profiles. In certain aspects, the anchors described herein can include bone screws or other bone fixators or connectors. Examples of bone screws and/or dimensional aspects of the holes 60, 70 are described in U.S. patent application Ser. No. 16/298,339, previously incorporated by reference herein.

As shown in FIG. 2, the first adjustment rod 40 can include two distinct portions 40A, 40B, which can be telescopically actuated to apply translational pressure on the patient's bone. For example, portions 40A, 40B of the first adjustment rod 40 can be configured to translate telescopically, e.g., as described with reference to FIGS. 1-5 of U.S. patent application Ser. No. 16/298,339, previously incorporated by reference herein. In certain cases, the drive mechanism 50 includes at least two distinct drivers 100, 110 that are configured to be actuated by an external control device. In some cases, the external control device includes an external actuator for communicating with the drive mechanism 50 from a location external to the patient's body, e.g., a magnetic controller and/or other wireless controller. In particular cases, the first driver 100 is configured to control translation of the first adjustment rod 40. In certain implementations, the first driver 100 includes a gear module (e.g., with planetary gears) 120 for driving movement of the portions 40A, 40B of the first adjustment rod 40. In some examples, the gear module 120 is coupled with a magnetic actuator (e.g., permanent magnet) 130 that is rotationally coupled to the gear module 120. However, other driving elements are suitable in place of a magnet. For example, in addition to or instead of the magnet-based driving, one or more of the driving elements can take the form of an implanted electric motor. The implanted electric motor can be powered by an external power source (e.g., via a radiofrequency link, via an inductive connection, or via another technique). The implanted electric motor can be powered by an implanted power source (e.g., a battery, which may be charged by the external power source). The implanted power source may be within the implant (e.g., within a housing thereof) or separate from the implant and coupled to the implant via a cable. In certain cases, the gear module 120 is mounted with radial bearings and/or thrust bearings to a lead screw 140 that drives translation of the portions 40A, 40B of the first adjustment rod 40. As noted herein, driver 100 can be configured to control translation of the first adjustment rod 40.

In particular cases, the second driver 110 is also configured to be actuated by an external control device. In certain cases, second driver 110 is configured to control rotation of the first adjustment rod 40. According to some examples, the second driver 110 is coupled with an additional magnetic actuator 150 for actuating the second driver 110. In various implementations, drivers 100, 110 are coupled to the housing 20 at distinct locations, for example, to resist unintentional actuation of at least one of the drivers. In certain cases, the magnetic actuators 130, 150 are physically separated (e.g., axially, along axis A) to mitigate unintentional triggering one of the actuators via a magnetic field, e.g., from the external controller. In certain implementations, the magnetic actuator 130 is axially interposed between the first driver 100 and the second driver 110 within the housing 20.

The second driver 110 can be coupled with a distal end 160 of the first adjustment rod 40, and can include a gear module 170 for driving rotation of the first adjustment rod 40. In particular cases, the gear module 170 is coupled with a mount 180 extending axially from the distal end 160 of the first adjustment rod 40. In practice, a change in magnetic field can trigger the actuator 150 to rotate, thereby driving the gear module 170 to cause rotation of the mount 180 (and coupled first adjustment rod 40). Accordingly, a user can control translation of the first adjustment rod 40 by actuating the magnetic actuator 130, and control rotation of the first adjustment rod 40 by separately actuating the magnetic actuator 150.

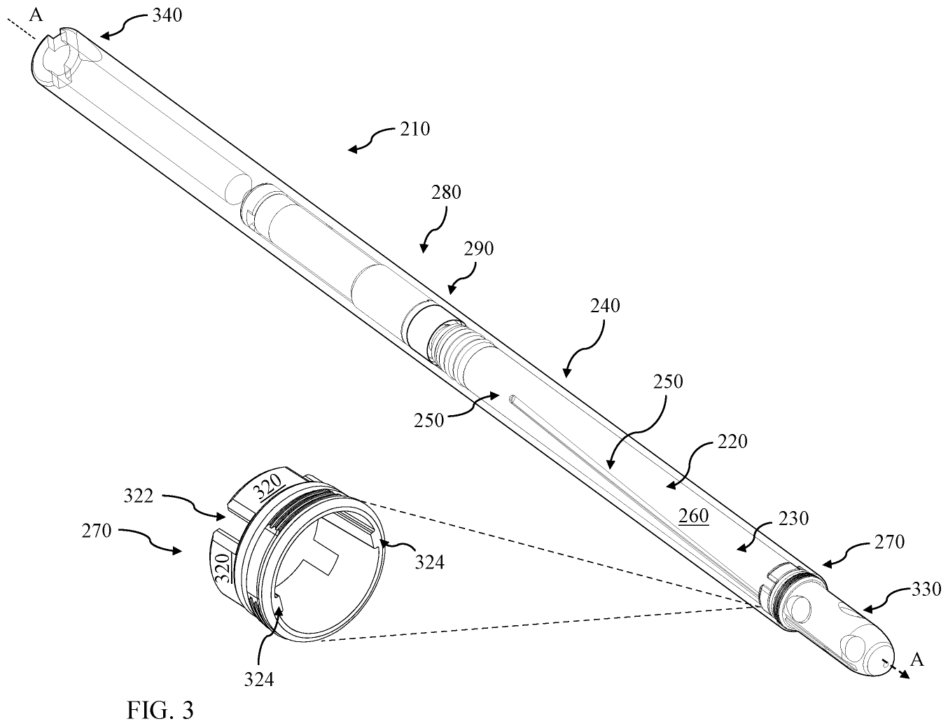

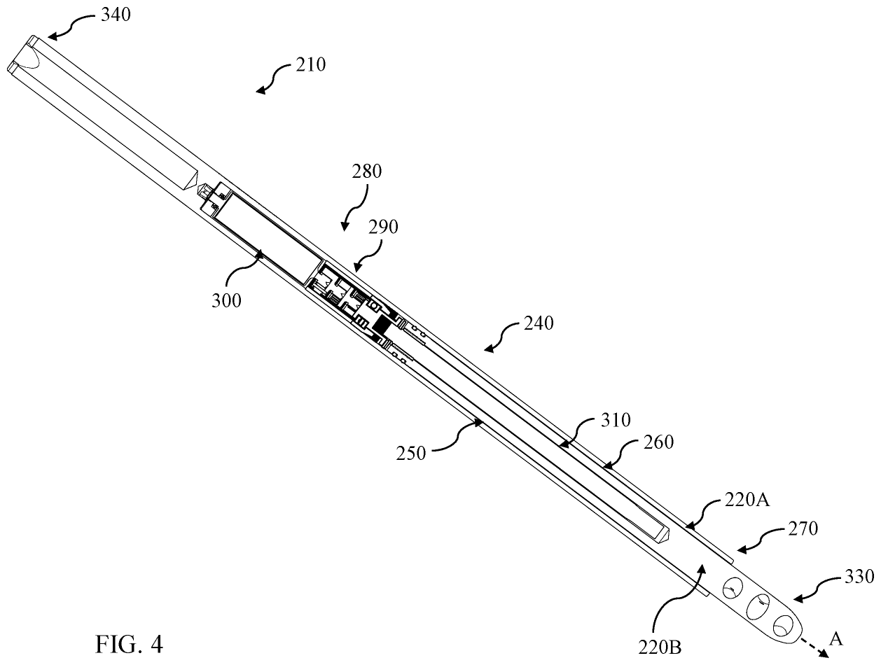

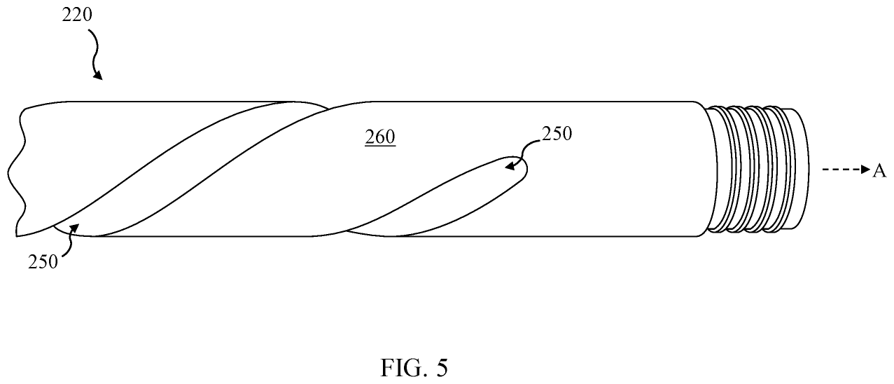

FIG. 3 shows an additional implementation of an implant 210, including a first adjustment rod 220 that has at least one helical groove 250. FIG. 4 is a cross-sectional depiction of the implant 210 in FIG. 3. FIG. 5 shows a close-up perspective view of the adjustment rod 220 according to certain implementations. In these implementations, similar to implant 10, a housing 240 at least partially contains the adjustment rod 220, which is configured to both translate axially and rotate about, the primary axis (A) of the housing 240. In certain cases, the adjustment rod 220 includes a plurality of helical grooves 250 along an outer surface 260 of the adjustment rod 220. In certain cases, the adjustment rod 220 has two helical grooves 250 along the outer surface 260, and in additional cases, the adjustment rod 220 has three or more helical grooves 250 along the outer surface 260. In various implementations, as illustrated in FIG. 3, the housing 240 includes a rotation control lug 270 for engaging the helical groove(s) 250. In certain cases, the rotation control lug 270 engages the helical groove(s) 250 such that for a portion of an axial extent of translation (along axis A) of the adjustment rod 220, the rotation control lug 270 causes the adjustment rod 220 to rotate about the primary axis A. In certain embodiments, the housing 240 includes a driver 280 including a gear module 290 coupled (e.g., rotationally) with a magnetic actuator (e.g., permanent magnet) 300 or other driving element. In certain cases, the gear module 290 is mounted with radial bearings and/or thrust bearings to a lead screw 310 that drives translation of portions 220A, 220B of the adjustment rod 220. As described with reference to driver 100 (FIGS. 1 and 2) herein, driver 280 can be configured to control translation of the adjustment rod 220.

In contrast to the embodiments shown and described relative to FIGS. 1 and 2, rotation of the adjustment rod can be controlled by the interaction between the helical groove(s) 250 and the rotation control lug 270. In certain examples, the rotation control lug 270 is an insert mounted within the housing 240, while in other examples, the rotation control lug 270 (and features thereof) are integral with an inner surface of the housing 240 that interfaces with the adjustment rod 220. An example rotation control lug 270 is shown in isolation in FIG. 3. In a particular example, the rotation control lug 270 includes at least one tab 320 that is configured to mate with helical groove(s) 250 to cause rotation of the adjustment rod 220 as it is translationally driven along axis A by driver 280. That is, when driven by the driver 280, the adjustment rod 220 is configured to translate and rotate simultaneously. In one non-limiting example, the rotation control lug 270 includes two distinct tabs 320 configured to mate with two distinct helical grooves 250 on the outer surface 260 of the adjustment rod 220, causing rotation of the adjustment rod 220 as it translates relative to the housing 240. In various implementations, the tabs 320 are separated (e.g., circumferentially) by spaces or gaps 322, and include protrusions 324 that extend axially (e.g., parallel to axis A) and engage helical grooves 250. Similarly to the implant 10, implant 210 can include holes 330 at a distal end of the implant 210 and holes 340 at a proximal end of the implant 210 for passing an anchor with which to secure the implant to the patient (e.g., to bone).

In a particular example, the helical groove(s) 250 has a dimension defined by a patient adjustment profile for the patient's body. For example, the helical groove(s) 250 can be customized for the patient, in terms of one or more physical characteristics such as groove diameter, pitch, grooves per centimeter, etc. In a particular example, the pitch of grooves 250 and number of grooves per centimeter are adjustable based on a patient adjustment profile. In such cases, a method of forming the adjustment rod 220 includes receiving at least one patient adjustment profile characteristic and assigning a value to at least one of groove pitch or grooves per centimeter in the manufacture of an adjustment rod (e.g., adjustment rod 220). In certain cases, the patient adjustment profile characteristic includes at least one of: a total translation distance, a total rotation degree, a rate of translation, a rate of rotation, a ratio of translation to rotation, or an adjustment period. The patient adjustment profile characteristics can be defined manually by a healthcare professional. In addition or instead, surgery planning software can be used to plan the patient adjustment profile characteristics. Once planned, the patient adjustment profile characteristics can be used to manufacture a custom helical groove 250 for the patient or to assist a surgery team in selecting an implant having a sufficiently close helical groove 250 to the determined characteristics.

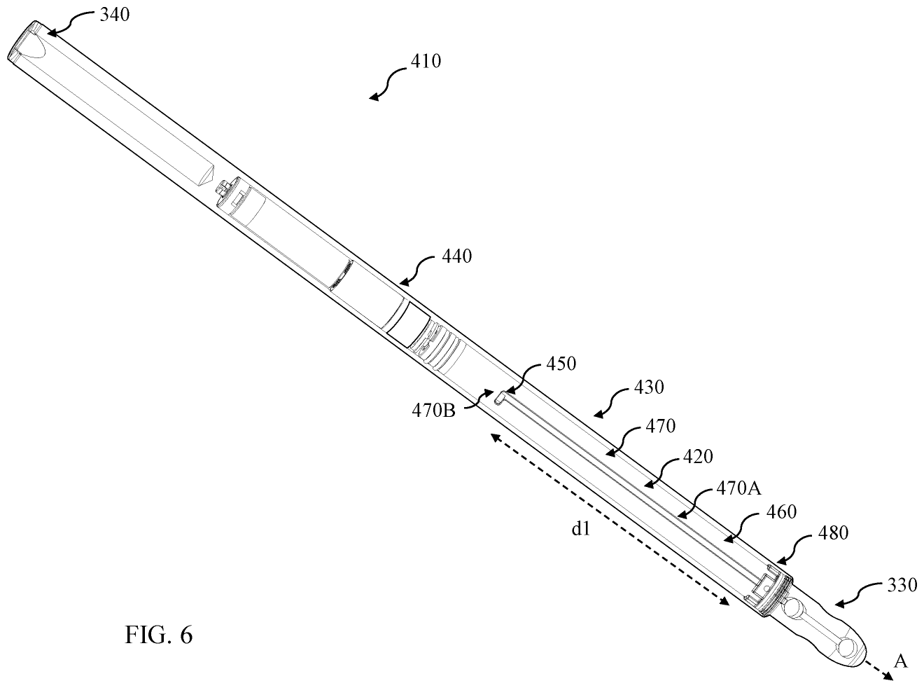

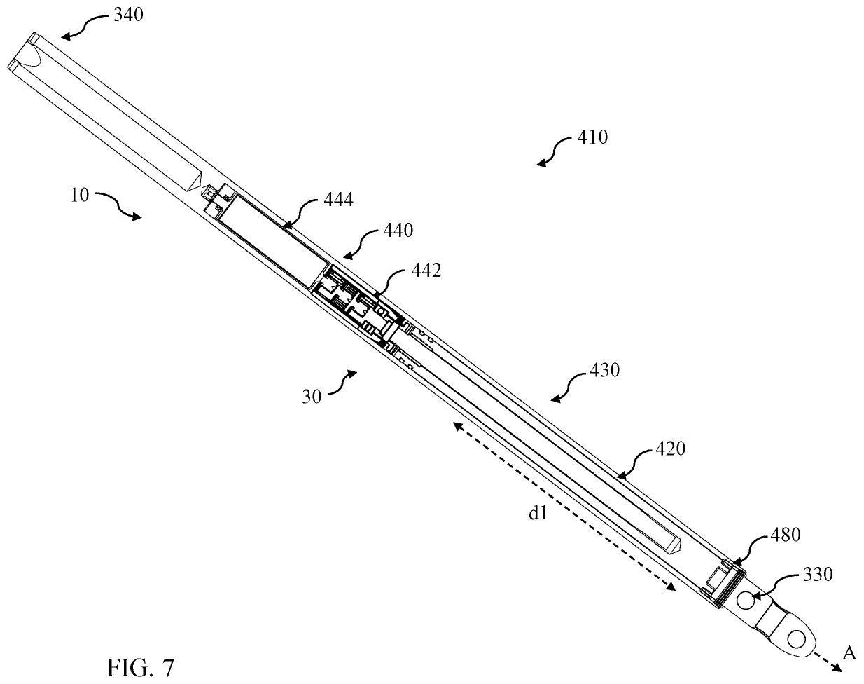

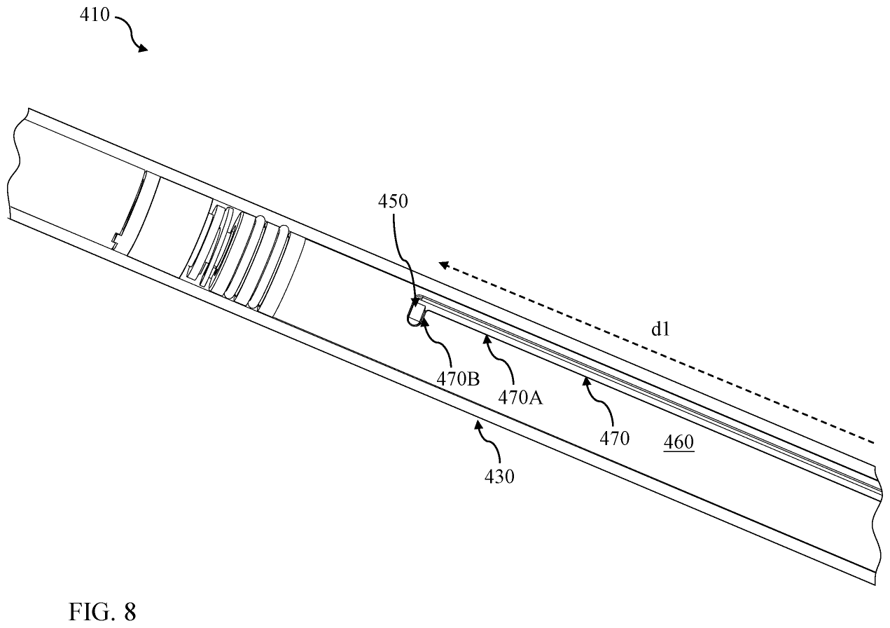

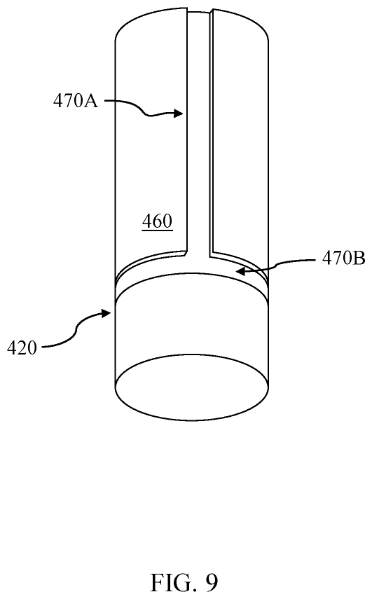

FIG. 6 shows a perspective view of an additional implant 410 according to various implementations. FIG. 7 shows a cross-sectional view of the implant 410, and FIG. 8 illustrates a close-up perspective view of a portion of the implant 410. Similar to additional implementations of implants herein, implant 410 can include a first adjustment rod 420 within a housing 430 that is configured to enable moving a patient's bone. In contrast to certain other implementations of implants herein, implant 410 includes an adjustment rod 420 that is configured to separately translate and rotate in response to driving by a drive mechanism 440 (e.g., having a driver including a gear module 442 and connected actuator 444 such as a magnetic actuator). That is, the adjustment rod 420 is configured to translate and rotate in separate processes in order to aid in bone movement. In particular cases, implant 410 is configured to translate a first distance (di) along axis (A), and after translating for the entirety of the first distance (di), is then free to rotate at least partially about the primary axis A. In various implementations, the adjustment rod 420 includes a radially extending tab 450 (FIG. 8) and the housing 430 includes a sleeve 460 with an axially extending slot (or, “slot”) 470 for engaging the radially extending tab 450. That is, the slot 470 engages the tab 450 to limit rotation along only a portion of the axial extent of translation of the adjustment rod 420, e.g., only along the first distance (di). In certain implementations, the tab 450 can be retractable, e.g., to control interfacing with the slot 470. In other cases, the tab 450 is permanently protruding, i.e., not retractable. In certain cases, the slot 470 has an axially extending section 470A that permits axial movement of the adjustment rod 420 (by limiting movement of the tab 450) and a circumferentially extending section 470B that permits rotation of the adjustment rod 420. In some cases, the circumferentially extending section 470B is continuous with the axially extending section 470A. In additional implementations, the slot 470 can be open-ended where the circumferentially extending section 470B is located, for example, such that it does not limit circumferential (rotational) movement of the tab 450. In certain cases, the implant 410 also includes a rotation control lug 480 that can have similar features as the rotation control lug 270 shown and described with reference to implant 210. In such cases, the rotation control lug 480 can act as a rotation limiter when the tab 450 of the adjustment rod 420 is not engaged with the axially extending section 470A of the slot 470.

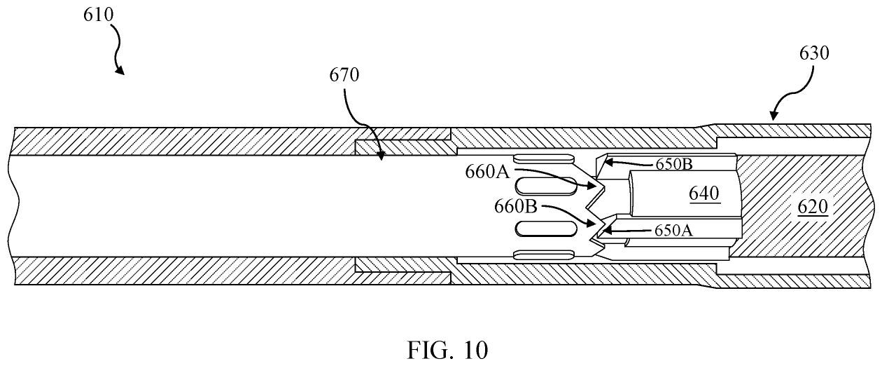

FIG. 10 illustrates another implementation of an implant 610 that includes a first adjustment rod 620 at least partially contained in a housing 630. In these implementations, the adjustment rod 620 includes a cam member 640 that has a set of teeth 650 for engaging complementary teeth 660 in the housing 630. In these implementations, the adjustment rod 620 is rotationally limited by interaction of the teeth 650 in the cam member 640 with the teeth 660 in the housing 630. For example, the interaction of teeth 650 with teeth 660 can provide a self-locking, or anti-back-rotation feature that permits an amount of rotation but prevents additional rotation or back-rotation in some cases. In a particular implementation, as illustrated in FIG. 10, the complementary teeth 660 in the housing 630 are axially offset to enable incremental translational adjustment of the first adjustment rod 620. For example, neighboring teeth 660A, 660B in the complementary teeth 660 can be axially offset (i.e., at distinct axial locations, relative to axis A) to enable incremental translation adjustment of the adjustment rod 620. In further cases, three or more neighboring teeth 660 can be axially offset to provide at least three levels of axial positioning for the teeth 650. In particular cases, the incremental translation adjustment is a stepwise translation and rotation of the adjustment rod 620, e.g., as driven by a drive mechanism 670. In certain cases, the drive mechanism 670 includes at least one spring and a gear pack (not shown) for controlling rotation of the adjustment rod 620. In use, the drive mechanism 670 is configured to actuate axial movement of the adjustment rod 620, e.g., in a pulsed or repeatable driving motion, and as teeth 650 disengage with complementary teeth 660, the adjustment rod 620 rotates to enable engagement of a given tooth 650A, 650B, etc. with the neighboring complementary tooth or teeth 660A, 660B, etc.

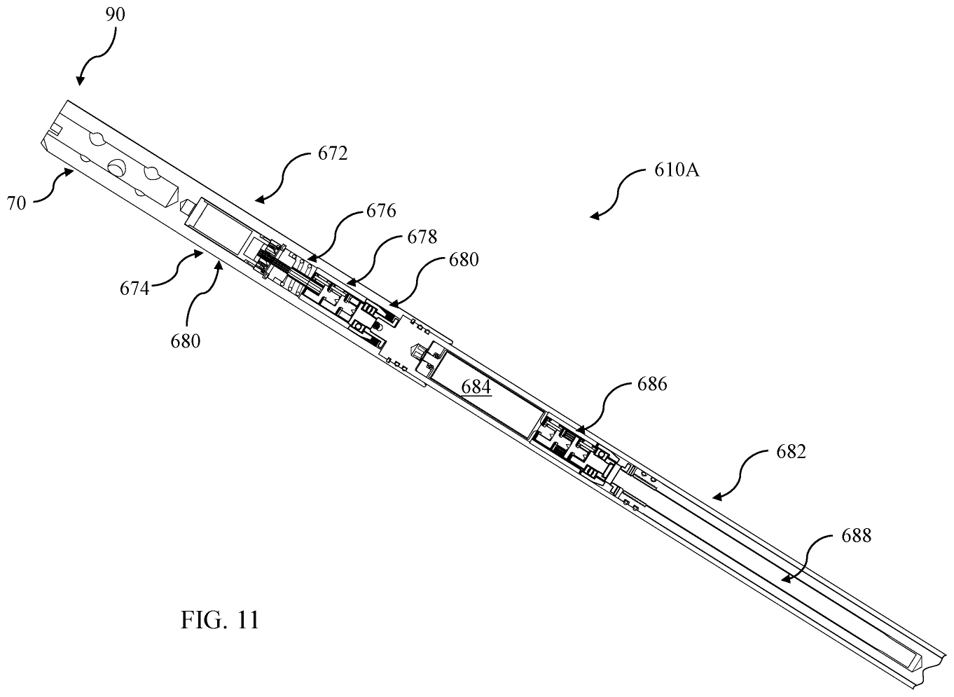

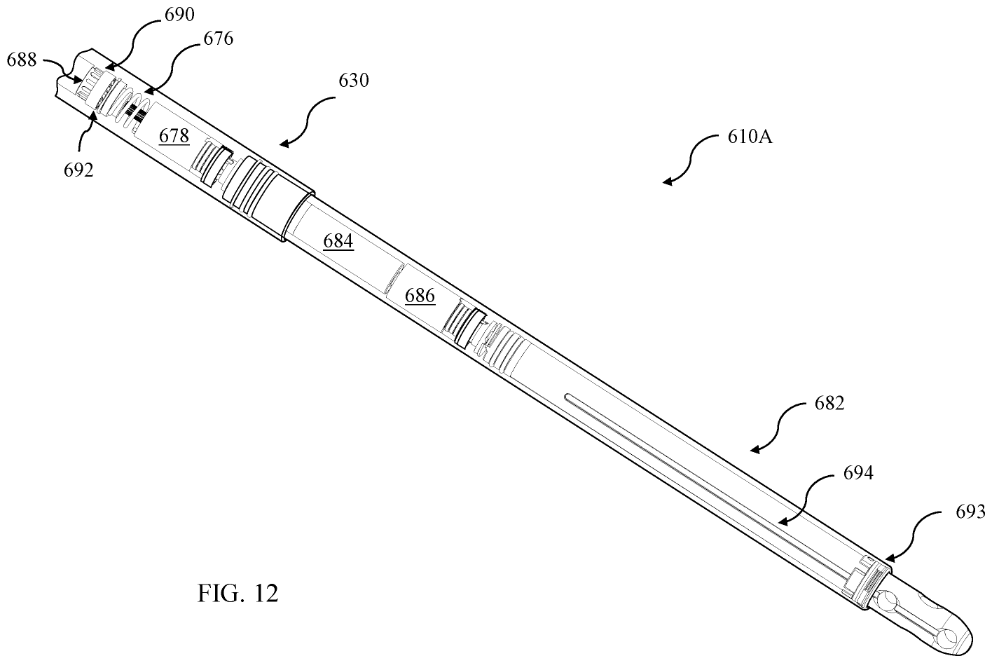

FIG. 11 shows a cross-section of a variation of an implant 610A according to certain implementations, and FIG. 12 shows a partially transparent perspective view of the implant 610A. In these implementations, a drive mechanism 672 includes a magnetic driver 674 coupled with a spring 676 and a gear pack 678 for axially driving a sub-housing 680 that is coupled with an adjustment rod 682 (including an associated magnetic driver 684, connected gear pack 686 and lead screw 688). In this example implementation, actuation of the magnetic driver 674 (e.g., via an external control device) drives the spring 676 and moves the sub-housing 680 axially. As shown in FIG. 12, driving the sub-housing 680 axially causes corresponding sets of teeth 688, 690 on the sub-housing 680 and a cam member 692 on the gear pack 686, respectively, to control rotation of the gear pack 686 and the coupled adjustment rod 682. For example, as the spring 676 is incrementally actuated to drive the sub-housing 680 axially, the teeth 690 on the cam 692 engage successive sets of teeth 688 on the sub-housing 680, e.g., to enable rotation of the gear pack 686 and the connected adjustment rod 682. In certain cases, as shown in FIG. 12, the implant 610A can include a rotation control lug 693 (similar to rotation control lug 270) that enables the adjustment rod 682 to rotate and for internal tabs on the lug 693 to engage distinct axially extending slots 694 on the rod 682 (or vice versa) to prevent undesirable back-rotation.

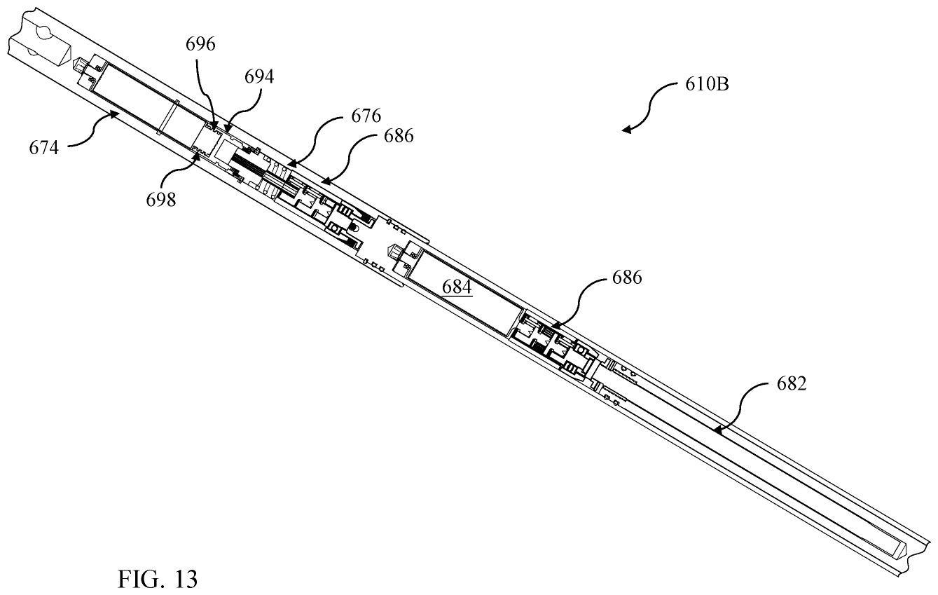

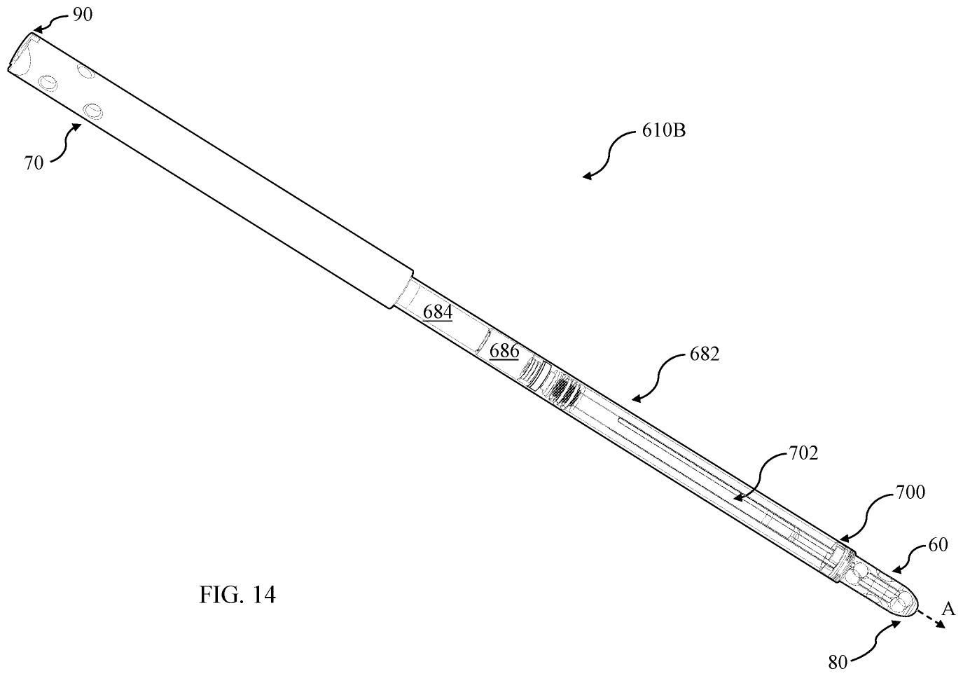

FIG. 13 shows a cross-section of a variation of an implant 610B according to certain implementations, and FIG. 12 shows a partially transparent perspective view of the implant 610B. In this embodiment, rotation of the gear pack 686 (and thereby, associated rotation of the adjustment rod 682) can be achieved by actuation of the magnetic driver 674 to drive spring 676. In such cases, the spring 676 drives rotation of a cam member 694 that has a set of threads 696 for engaging corresponding threads 698 coupled with the driver 674. As the axial force of the spring 676 is applied to the cam member 694, the cam member 694 translates that axial force into rotational motion on the gear pack 686. In certain cases, the threads 696 can include notched threads that have an anti-back-rotation feature. In other implementations, as shown in FIG. 14, the implant 610B can include a rotation control lug 700 (similar to rotation control lug 270) that enables the adjustment rod 682 to rotate and for internal tabs on the lug 700 to engage distinct axially extending slots 702 on the rod 682 and prevent undesirable back-rotation.

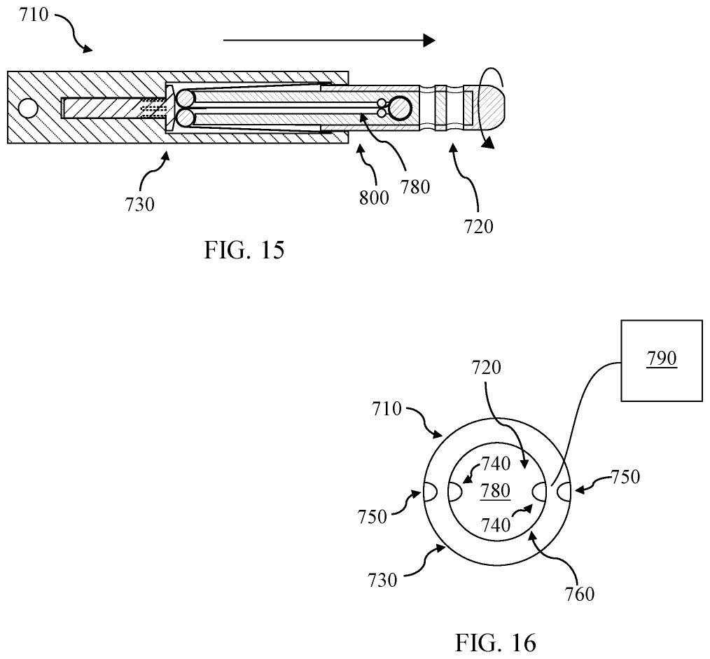

FIG. 15 illustrates another implementation of an implant 710 configured to move bone in a patient's body. FIG. 16 shows a cross-section of the implant 710 through a housing 730. In these cases, implant 710 includes a first adjustment rod 720 at least partially contained in housing 730. In these cases, the first adjustment rod 720 includes a set of (at least one) retractable tabs 740 that complement at least one mating feature 750 in the housing 730. For example, the retractable tabs 740 are configured to retract and extend (radially, relative to primary axis A) in order to engage mating feature(s) 750 in the housing. FIG. 16 shows a cross-section through the housing 730 and the rod 720, illustrating an example configuration with two tabs 740 and two mating features 750. In this configuration, interaction between the retractable tab(s) 740 and the mating feature(s) 750 can limit rotation of the rod 720 within the housing 730. For example, the tab(s) 740 and mating feature(s) 750 are configured to be aligned circumferentially to prevent rotation of the rod 720 when engaged. When the tab(s) 740 are retracted (as shown in FIG. 16), the rod 720 has freedom to rotate, at least partially, within the housing 730. In certain implementations, the retractable tabs 740 can be retracted or extended by means of a rotatable body (e.g., an intermediate body) 760 with a set of apertures (not shown) for accommodating the tabs 740. The tabs 740 can be radially actuatable, e.g., to retract or extend in response to rotation of the body 760. In certain cases, the tabs 740 include a taper, rounded portion or an arc that enables the body 760 to interface with the tabs 740 and cause them to retract when not aligned with the apertures. In other implementations, the tabs 740 are configured to retract into a core section 780 of the adjustment rod 720. In any case, when extended, each tab 740 is configured to engage a mating feature 750 to limit rotation of the adjustment rod 720 relative to the housing 730. In contrast, when retracted, the tab(s) 740 enable rotation of the adjustment rod 720 relative to the housing 730.

In certain cases, retraction and/or extension of the tabs 740 (e.g., by directly controlling retraction of the tabs 740 and/or controlling rotation of the intermediate body 760) is controlled with an electronic controller 790 that is coupled with the adjustment rod 720. In certain cases, the controller 790 is coupled with a solenoid-based actuator to actuate movement of the tabs 740 and/or movement of the intermediate body 760. In some cases, the controller 790 is separate from other control components in the drive mechanism 710 (e.g., components driving axial translation of the adjustment rod 720). In other cases, the controller 790 can be integrated into a housing with the drive mechanism 710. In certain implementations during use, the proximal end 800 (FIG. 15) of the adjustment rod 720 engages a lead screw (not shown) and is configured to move with the lead screw. In these cases, while the retractable tab(s) 740 engage the mating feature(s) 750, the adjustment rod 720 is configured to translate during movement of the lead screw. Additionally in these cases, while the retractable tab(s) 740 are retracted, the adjustment rod 720 is configured to rotate during movement of the lead screw.

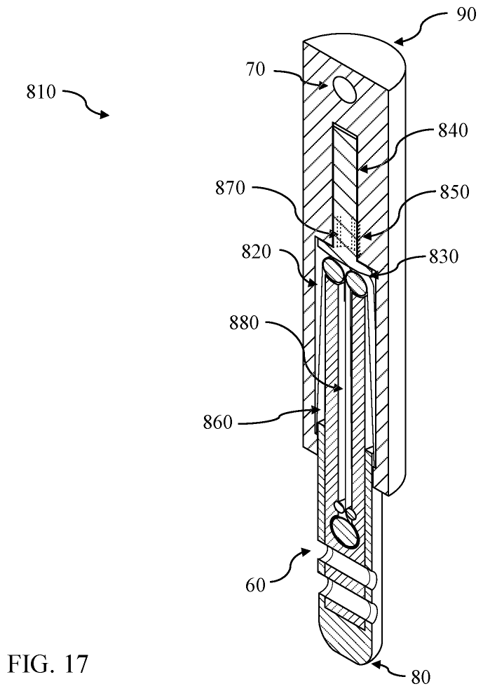

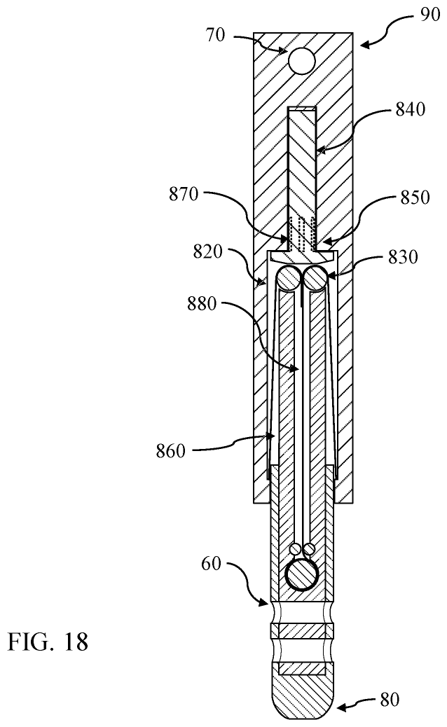

FIGS. 17 and 18 illustrate another implementation of an implant 810 for moving bone in a patient's body. FIG. 17 shows a cut-away perspective view of the implant 810, while FIG. 18 shows a cross-section of the implant 810. In certain cases, the implant 810 includes a drive mechanism 820 with a pulley plate 830 and a gear pack 840. In certain cases, the pulley plate 830 is coupled with a proximal end 850 of a first adjustment rod 860 (e.g., similar to one of the adjustment rods described herein) to control translation of the first adjustment rod 860. In certain cases, the gear pack 840 drives the pulley plate 830, and itself can be driven magnetically or by any other drive mechanism described herein with reference to gear packs. In some cases, the gear pack 840 engages complementary teeth (or gears) 870 on the pulley plate 830 to enable rotation of the pulley plate and the adjustment rod 860. In certain examples, the gear pack 840 includes at least one belt 880 coupled with an actuator for driving the pulley plate 830.

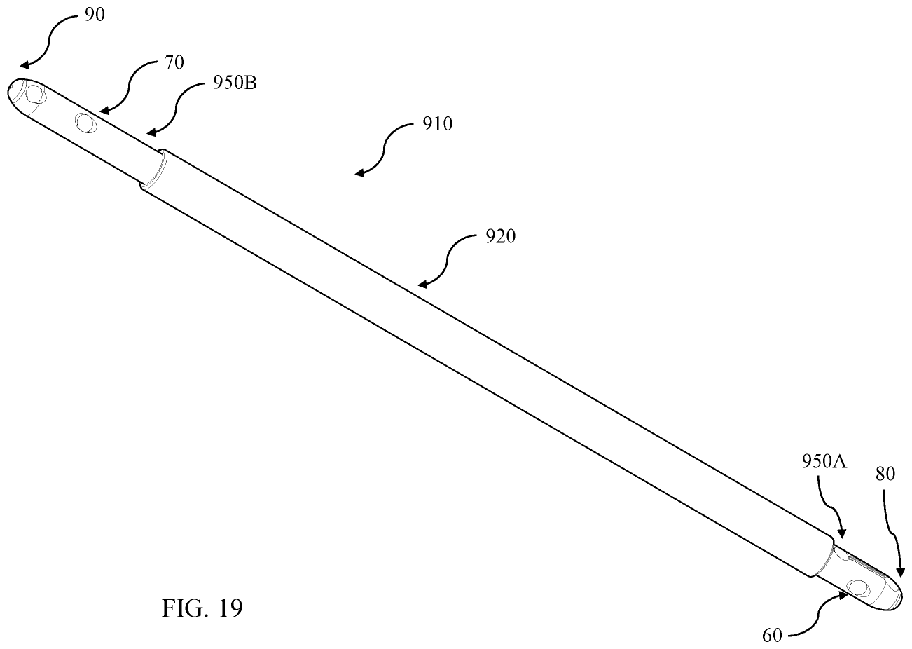

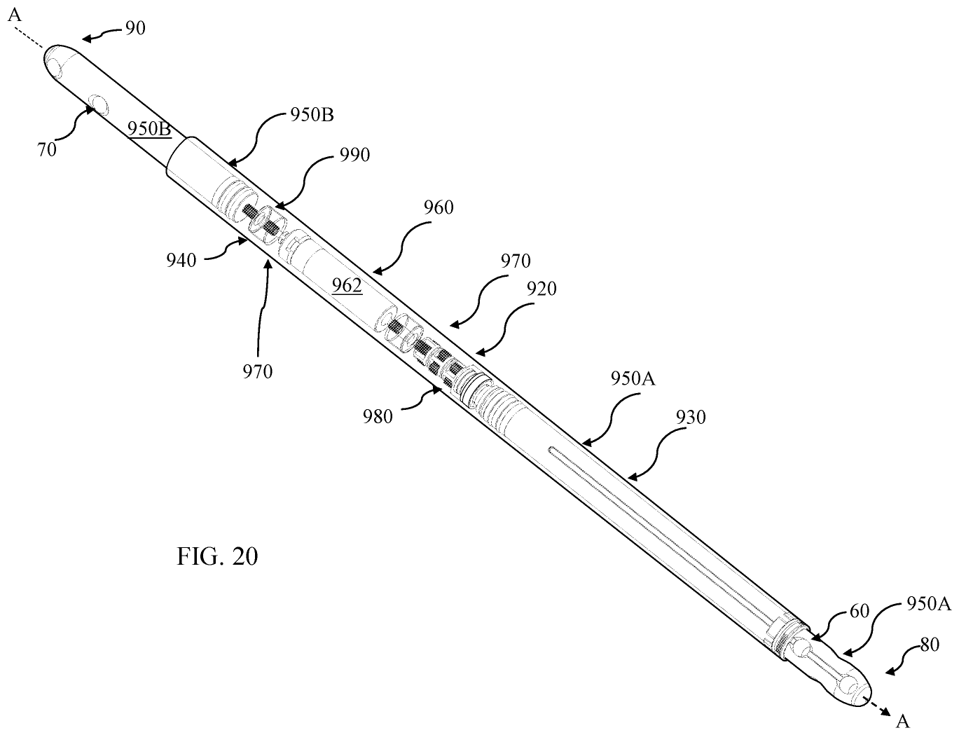

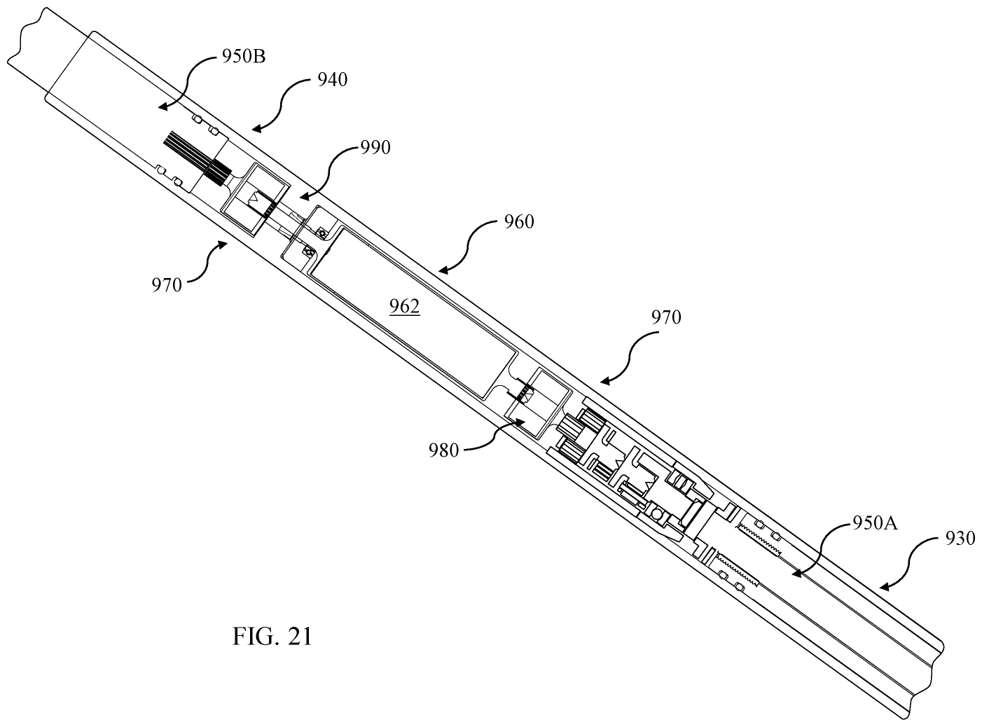

FIG. 19 shows another implementation of an implant 910 for moving bone in a patient's body in a perspective view, and FIG. 20 shows a cross-section of the implant 910. FIG. 21 is a close-up view of FIG. 20. In these implementations, the implant 910 includes an implantable biocompatible housing 920 that includes a first cavity 930 and a second cavity 940. The implant 910 also includes a set of adjustment rods 950 that are at least partially contained within the first and second cavities 930, 940. The implant 910 further includes a drive mechanism 960 configured to drive the adjustment rods 950 to enable both translation axially relative to the housing 920 and rotation about a primary axis (A) of the housing 920. Implant 910 can include various features and components with similar function (and in certain cases, form) as the implant 10 (FIG. 1). However, in certain cases, implant 910 includes at least two distinct adjustment rods for enabling both translational (or, axial) adjustment and rotational adjustment of the patient's bone. In a particular implementation, the set of adjustment rods 950 includes a first adjustment rod 950A at least partially contained in the first cavity 930 and a second adjustment rod 950B at least partially contained in the second cavity 940. In such cases, the drive mechanism 960 can include a clutch implant 970 that is configured to control adjustment of both the first adjustment rod 950A and the second adjustment rod 950B. In a particular implementation, the clutch implant 970 is positioned coaxially with the first adjustment rod 950A and the second adjustment rod 950B. However, in other implementations the clutch implant 970 can be positioned off-axis relative to the adjustment rods 950A, 950B. In some implementations, e.g., as illustrated in FIGS. 20 and 21, the clutch implant 970 is positioned between the first and second adjustment rods 950A, 950B.

According to certain implementations, the clutch implant 970 includes a first gear set 980 for controlling translation of the first adjustment rod 950A. In such cases, the clutch implant 970 can further include a second gear set 990 for controlling rotation of the second adjustment rod 950B. In some examples, the clutch implant 970 includes a magnetic clutch and/or an electronic clutch that is configured to be actuated by an external control device (such as an external actuator for communicating with the drive mechanism 960 from a location external to the patient's body, e.g., a magnetic controller and/or other wireless controller). In particular cases, e.g., as described with reference to implant 10, the drive mechanism 960 in implant 910 can include a magnetic actuator 962 configured to be actuated by a magnetic field external to the patient's body.

As with the other implants shown and described herein, implant 910 can be configured for intramedullary placement in a patient, e.g., to aid in treatment of a limb length discrepancy or a bone defect in the patient's body. In certain cases, the implants described and depicted herein can be used in a method of intramedullary adjustment of a patient's bone, e.g., by inserting the implant(s) into the patient's body and by actuating the implant(s) using a controller such as an external control device.

Any implant described herein can be part of an implantable adjustment system that incorporates an external remote controller (ERC) or other external control device. In certain cases, the ERC can include a magnetic handpiece, a controller (or control box, e.g., with a processor), and a power supply. In additional implementations, the ERC or other external control device can include an interface such as a user interface for enabling a medical professional to interact with the system including implant(s) described herein. Additional details of an ERC and interaction with implants are described in U.S. patent application Ser. No. 16/298,339, previously incorporated by reference herein.

Even further, the implants, associated systems and controllers can include a communication system for connecting devices (e.g., via wireless or hard-wired means), or integral with particular devices (e.g., ERC). The communication system can include a number of hard-wired and/or wireless communication systems, with certain wireless systems configured to communicate over Bluetooth, Bluetooth Low Energy (BLE), radio frequency (RF), Wi-Fi, and/or ultrasound. In additional implementations, the communication system can include an independent subscriber identity module (SIM) assigned to each implant. In further cases, the communication system is configured to communicate wirelessly with a remote control system and/or data gathering/analysis platform, e.g., via a cloud-based communication protocol.

In particular cases, each implant is individually programmable to control an amount of the adjustment of the patient's bone. For example, implants described herein may each include an individually programmable or adjustable component (e.g., programmable controller and/or gear ratio, thread pitch and/or count, etc.) to control the amount of adjustment of the patient's bone. In certain cases, distinct implants in a system can be programmed or otherwise designated to perform distinct adjustments.

In additional cases, the controller(s) described herein includes a smart device (e.g., smart phone, smart watch, tablet, etc.) configured to operate a control platform for adjusting the implants. In these instances, the control platform can include a software application (or “app”) configured to execute or otherwise run at a controller (e.g., ERC) for enabling control of one or more implants. According to certain implementations, the control platform enables control functions for one or more implants from a remote physical location relative to device 100. For example, the control platform can enable connection (e.g., network-based and/or cloud-based connection) between a system including the implant(s) described herein and a remote user such as a medical professional.

In all implementations described herein, the implant(s) can further include a feedback system in communication with one or more control devices (e.g., ERC and/or software application running control program). In certain cases, the feedback system provides feedback on a force response to the adjustment of the length of a given adjustment rod and/or rotation of a given adjustment rod. In certain cases, the feedback system includes a sensor onboard the implant, e.g., a sensor that is integrated with or coupled with the housing. Non-limiting examples of sensors can include a load cell, a piezo (piezoelectric) sensor, or an imaging sensor (e.g., optical sensor such as a camera, or an ultrasound sensor). Additional sensors that can be integrated in, or otherwise form part of the feedback system can include position and/or speed sensors (e.g., gyroscope/magnetometer, or inertial measurement unit (IMU)), temperature sensors and/or humidity sensors. In certain cases, the feedback system provides instructions to the controller (e.g., ERC) to modify actuation of a given implant based on the feedback on the force response.

In still further implementations, the sensor(s) in the feedback system described herein can be configured to provide data about a load exerted on an adjustment element, and/or a load exerted by the adjustment element on the patient's bone. The sensor(s) can also provide data about a tensile load between the implants and bone. In certain implementations, both torque and compression data are recorded by sensor(s) and provided to the feedback system for analysis and/or action (e.g., to adjust adjustment instructions). It is understood that torque and/or compression data detected by sensors, can represent an inferred or correlated indicator of the torque and/or compression applied to a device or component not physically in contact with the sensor. For example, the sensor on an instrument can be configured to detect torque at the instrument, while that torque is being translated to a driven element in contact with the distal end of the instrument. Similarly, the sensor on an instrument can detect compression at the instrument, while that compression is being translated to an external component, e.g., a driven element.

In additional implementations, one or more device components described herein, e.g., driving elements in implants, can be communicatively coupled with a navigation system that is configured to detect a position of the instrument(s). In one example, the control unit (e.g., ERC) can include or otherwise communicate with a navigation system in order to provide navigation information about a position of instruments. For example, the navigation system can include an optical tracking system such as a camera or laser-based tracking system, a Global Positioning System (GPS), an inertial measurement unit (IMU), an ultrasound based measurement system, other kinds of position systems, or combinations thereof. In certain cases, the navigation system is configured to determine a distance moved by the instrument when the instrument changes position, which the navigation system communicates to the control unit (e.g., for processing by the feedback system). One or more components of a navigation system can be located within or otherwise integrated with a housing that is mounted to or otherwise coupled with one or more of the device components.

In certain cases, the feedback system, or functions thereof, can be integrated into a control unit and/or a controller as described herein. In particular cases, the feedback system is part of a software application and is configured to determine what, if any, force adjustment should be made at a given implant based on the force feedback. In some examples, the feedback system includes a model that correlates force response and force applied during adjustment of the length of an implant. The model can be based at least in part on historical data from a set of implants in distinct bone fixation devices, e.g., similar to implant(s) described herein. According to various implementations, the model can be updated periodically, or on a continuous basis, to provide additional data about force response as compared to force applied in one or more implants. In certain cases, a version of the model can be downloaded or otherwise stored locally at one or more control units and/or controllers and periodically updated, e.g., via a cloud-based or other network-based software update. This approach can reduce the computational and/or storage requirements at control unit(s) and controller(s) that may be local to the implant(s).

In additional implementations, the feedback system is configured to provide postoperative data, post-adjustment data, and analysis of alignment procedure and/or device usage, e.g., to enhance future procedures and/or diagnose inefficiencies in a past procedure. In certain implementations, the feedback system is configured to update the control instructions for control unit(s) based on identified inefficiencies or errors in adjustment quantities (e.g., lengthening, rotation) and/or device usage during/after a given procedure. In particular implementations, the feedback system includes a logic engine configured to modify instructions iteratively, e.g., on a procedure-by-procedure or patient-by-patient basis.

While many examples are described in the context of intramedullary nails, the technologies and components described herein can be adapted for use in other applications. For instance, a rod configured to treat scoliosis (e.g., as described in U.S. application Ser. No. 11/172,678, which was filed Jun. 30, 2005, and which is hereby incorporated herein by reference in its entirety for any and all purposes), can be modified based on examples described herein. For instance, the rod configured to treat scoliosis can be configured to support both rotation and distraction.

As another example, a intramedullary device for ankle fusion (e.g., as described in U.S. application Ser. No. 17/699,116, which was filed Mar. 19, 2022, and which is incorporated herein by reference for any and all purposes) can be modified based on examples described herein. For instance, the intramedullary device for ankle fusion can be configured to support both rotation and distraction.

As another example, an adjustable device for treating arthritis of the knee (e.g., as described in U.S. application Ser. No. 15/953,453, which was filed Apr. 15, 2022, and which is incorporated herein by reference for any and all purposes) can be modified based on examples described herein. For instance, the adjustable device for treating arthritis of the knee can be configured to support both rotation and distraction.

As another example, a bone transport device (e.g., as described in U.S. application Ser. No. 13/655,246, which was filed Oct. 18, 2012, and which is incorporated herein by reference for any and all purposes) can be modified based on examples described herein. For instance, the bone transport sled of the apparatus can be configured to support both rotation and translation.

Various additional aspects of the disclosure can include a method of intramedullary adjustment of a patient's bone using the implant(s) described herein. Using FIGS. 1 and 2 strictly for the simplicity of illustration, the method can include adjusting a patient's bone using an intramedullary implant such as implant 10 by: (i) coupling the implant 10 to the patient's bone (e.g., via bone screws or other fasteners at holes 60, 70); and (ii) actuating adjustment of the length of the implant 10 and/or actuating rotation of the implant 10 with an external control device (e.g., ERC or other remote controller). The adjusting of the length can occur before, during, or after adjusting of the rotation. In certain cases, after adjusting the patient's bone, a method can further include: (iii) decoupling the implant from the patient's bone (e.g., via bone screws or other fasteners at holes 60, 70).

In certain cases, a method can include imaging a bone connected with the implant(s) described and illustrated herein. For example, a method can include: (I) coupling or decoupling an implant (e.g., implant 10) with a patient's bone, and (II) imaging the bone with MRI and/or X-ray imaging after the coupling or decoupling. After imaging, the method can further include: (III) either (a) adjusting an already coupled implant (e.g., implant 10) or (b) decoupling the already coupled implant (e.g., implant 10) based on feedback from the imaging process.

As noted herein, the implants and associated methods described herein enable multi-modal adjustment, which can reduce time and complications associated with bone adjustment procedures. The various disclosed implementations can improve patient outcomes when compared with conventional implantable adjusters, for example, increasing adaptability in adjusting bone positioning, enhancing both intraoperative and postoperative engagement with the device. When compared with conventional approaches, the multi-modal (e.g., translational and rotational) adjustment implants described according to various implementations provide an efficient and simplified mechanism for bone adjustment. The implants described according to various implementations can also reduce health risks for patients when compared with conventional approaches, for example, allowing a single implant to perform functions conventionally performed by multiple implantable devices (with associated implant procedures).

The functionality described herein, or portions thereof, and its various modifications (hereinafter “the functions”) can be implemented, at least in part, via a computer program product, e.g., a computer program tangibly embodied in an information carrier, such as one or more non-transitory machine-readable media, for execution by, or to control the operation of, one or more data processing apparatus, e.g., a programmable processor, a computer, multiple computers, and/or programmable logic components.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a network.

Actions associated with implementing all or part of the functions can be performed by one or more programmable processors executing one or more computer programs to perform the functions of the calibration process. All or part of the functions can be implemented as, special purpose logic circuitry, e.g., an FPGA and/or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. Components of a computer include a processor for executing instructions and one or more memory devices for storing instructions and data.

In various implementations, components described as being “coupled” to one another can be joined along one or more interfaces. In some implementations, these interfaces can include junctions between distinct components, and in other cases, these interfaces can include a solidly and/or integrally formed interconnection. That is, in some cases, components that are “coupled” to one another can be simultaneously formed to define a single continuous member. However, in other implementations, these coupled components can be formed as separate members and be subsequently joined through known processes (e.g., soldering, fastening, ultrasonic welding, bonding). In various implementations, electronic components described as being “coupled” can be linked via conventional hard-wired and/or wireless means such that these electronic components can communicate data with one another. Additionally, sub-components within a given component can be considered to be linked via conventional pathways, which may not necessarily be illustrated.

Commonly labeled components in the FIGURES are considered to be substantially equivalent components for the purposes of illustration, and redundant discussion of those components is omitted for clarity.

While inventive features described herein have been described in terms of preferred embodiments for achieving the objectives, it will be appreciated by those skilled in the art that variations may be accomplished in view of these teachings without deviating from the spirit or scope of the invention. Also, while this invention has been described according to a preferred use in spinal applications, it will be appreciated that it may be applied to various other uses desiring surgical fixation, for example, the fixation of long bones.

Various example embodiments of devices (e.g., implants) and techniques for moving bone in a patient's body are described herein. In the interest of clarity, not all features of an actual implementation are necessarily described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The implants and related systems, program products and methods described herein boast a variety of inventive features and components that warrant patent protection, both individually and in combination.

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

A number of implementations have been described. Nevertheless, it will be understood that additional modifications may be made without departing from the scope of the inventive concepts described herein, and, accordingly, other implementations are within the scope of the following claims.

We claim:

1. An implant for moving bone in a patient's body, the implant comprising:
an implantable biocompatible housing having a first cavity;
a first adjustment rod at least partially contained within the first cavity; and
a drive mechanism configured to drive the first adjustment rod to enable both translation axially relative to the housing and rotation about a primary axis of the housing.

2. The implant of claim 1, wherein the drive mechanism comprises:
a first driver controlling translation of the first adjustment rod relative to the housing; and
a second driver controlling rotation of the first adjustment rod relative to the housing.

3. The implant of claim 1, wherein:
the first adjustment rod comprises at least one helical groove; and
the housing includes a rotation control lug for engaging the at least one helical groove, such that for at least a portion of an axial extent of translation of the first adjustment rod, the rotation control lug causes the first adjustment rod to rotate about the primary axis.

4. The implant of claim 3, wherein:
the rotation control lug includes at least one tab configured to mate with the at least one helical groove; and
the at least one helical groove has a dimension defined by a patient adjustment profile customized for the patient's body.

5. The implant of claim 1, wherein:
the housing includes a sleeve having an axially extending slot;
the first adjustment rod includes a radially extending tab engaging the axially extending slot to limit rotation along only a portion of the axial extent of translation of the first adjustment rod; and
a proximal end of the first adjustment rod includes at least one rotation limiting feature for limiting rotation while the radially extending tab is not engaged with the axially extending slot in the sleeve.

6. The implant of claim 1, wherein:
the first adjustment rod comprises a cam member having a set of teeth for engaging complementary teeth in the housing;
the first adjustment rod is rotationally limited by interaction of the set of teeth in the cam member with the complementary teeth; and
the set of complementary teeth in the housing are axially offset to enable incremental translation adjustment of the first adjustment rod.

7. The implant of claim 1, wherein:
the first adjustment rod comprises a set of retractable tabs that complement at least one mating feature in the housing;
when extended, the set of retractable tabs are configured to engage the at least one mating feature in the housing to limit rotation of the first adjustment rod; and
when retracted, the set of retractable tabs enable rotation of the first adjustment rod relative to the housing.

8. The implant of claim 7, further comprising:
an electronic controller coupled with the first adjustment rod to control at least one of retraction or extension of the set of retractable tabs,
wherein the drive mechanism is configured to drive the axial translation of the first adjustment rod.

9. The implant of claim 1, wherein the drive mechanism includes:
a pulley plate is coupled with a proximal end of the first adjustment rod to control translation of the first adjustment rod; and
a gear pack that engages complementary teeth or gears on the pulley plate to enable rotation of the pulley plate and the first adjustment rod.

10. The implant of claim 1, wherein:
the drive mechanism includes a magnetic actuator configured to be actuated by a magnetic field external to the patient's body; and
the implant is a rod configured to treat scoliosis, an intramedullary device for ankle fusion, an adjustable device for treating arthritis of the knee, or a bone transport device.

11. An implant for moving bone in a patient's body, the implant comprising:
an implantable biocompatible housing having at least one cavity and a second cavity;
a set of adjustment rods comprising:
a first adjustment rod at least partially contained within the first cavity; and
a second adjustment rod at least partially contained within the second cavity; and
a drive mechanism configured to drive the set of adjustment rods to enable both translation axially relative to the housing and rotation about a primary axis of the housing.

12. The implant of claim 11, wherein the drive mechanism includes a magnetic or electronic clutch positioned coaxially with and between the first and second adjustment rods and being configured to control adjustment of both the first adjustment rod and the second adjustment rod.

13. The implant of claim 11, wherein the drive mechanism includes a magnetic actuator configured to be actuated by a magnetic field external to the patient's body.

14. The implant of claim 11, wherein the drive mechanism is the sole drive mechanism of the implant.

15. The implant of claim 11, wherein:
the first or second adjustment rod each have at least one helical groove; and
the housing includes a rotation control lug for engaging the at least one helical groove, such that for at least a portion of an axial extent of translation of the rod having the groove, the rotation control lug causes the rod having the groove to rotate about the primary axis.

16. The implant of claim 15, wherein the groove is customized for the patient.

17. The implant of claim 11, wherein:
the first adjustment rod comprises a cam member having a set of teeth for engaging complementary teeth in the housing;
the first adjustment rod is rotationally limited by interaction of the set of teeth in the cam member with the complementary teeth; and
the set of complementary teeth in the housing are axially offset to enable incremental translation adjustment of the first adjustment rod.

18. The implant of claim 11, wherein:
the first adjustment rod comprises a set of retractable tabs that complement at least one mating feature in the housing;
when extended, the set of retractable tabs are configured to engage the at least one mating feature in the housing to limit rotation of the first adjustment rod; and
when retracted, the set of retractable tabs enable rotation of the first adjustment rod relative to the housing.

19. The implant of claim 18, further comprising:
an electronic controller coupled with the first adjustment rod to control at least one of retraction or extension of the set of retractable tabs,
wherein the drive mechanism is configured to drive the axial translation of the first adjustment rod.

20. The implant of claim 11, wherein the drive mechanism includes:
a pulley plate coupled with a proximal end of the first adjustment rod to control translation of the first adjustment rod; and
a gear pack that engages complementary teeth or gears on the pulley plate to enable rotation of the pulley plate and the first adjustment rod.
